(19) United States
(12) Patent Application Publication
(10) Pub. No.: US 2019/0374789 A1
(43) Pub. Date: Dec. 12, 2019

(54) UPCONVERSION FLUORESCENCE-BASED PHOTOBIOMODULATION

(71) Applicant: Photoprotective Technologies Incorporated, San Antonio, TX (US)

(72) Inventor: James M. Gallas, San Antonio, TX (US)

(73) Assignee: Photoprotective Technologies Incorporated, San Antonio, TX (US)

(21) Appl. No.: 16/438,433

(22) Filed: Jun. 11, 2019

Related U.S. Application Data
(60) Provisional application No. 62/683,496, filed on Jun. 11, 2018.

(51) Int. Cl. A61N 5/06; G02C 7/02; A61K 8/96

(52) U.S. Cl. CPC A61N 5/062; G02C 7/021; A61N 5/0616; A61N 2005/0659; A61N 2005/063; A61N 2005/0648; A61N 2005/0657; A61K 8/96

(57) ABSTRACT

An ophthalmic lens comprising a clear, non-tinted, solid plastic substrate and a dye suitable for undergoing fluorescence upconversion wherein said dye absorbs light in the near infrared region from 850 nm to 2500 nm and emit light in the shorter wavelength region from 650 nm to 850 nm thereby providing photobiomodulation to the eye.

### RELATED PRIORITY DATE APPLICATION

This application claims the benefit under 35 U.S.C. 119(e) of the United States provisional application No. 62/683,496 filed on Jun. 11, 2018.

### FIELD OF THE INVENTION

The present invention relates to the field of light filters that provide repair to the eyes and skin through the process of photobiomodulation. The source of the red and near infrared light associated with the process of photobiomodulation in this invention is fluorescence and, more specifically, the nature of the fluorescence is based upon the use of material that are able to undergo fluorescence by way of photon upconversion

### BACKGROUND OF THE INVENTION

Prior art includes the work of Gallas (U.S. Pat. No. 9,726,910). In that work a light filter, for example a sunglass lens, absorbs the UV or high energy visible wavelengths of sunlight, and part of this absorbed energy is then converted into the red and near infrared wavelengths of light associated with repair of the cells and tissue of humans and animals—rather than being completely converted into heat, as in the case of most sunglass lenses. In this process, called photobiomodulation (or PBM) there are certain wavelengths of the aforementioned red and near infrared light that are absorbed by cytochrome C which, in turn, energizes the cell and improves various functions of the cell resulting in the repair of the cells, for example the photoreceptors of vision and in the general improvement of the tissue—such as the skin.

Prior art also includes the work by Rudolph et. al. (Pub. No.: US 2013/0309184A 1). In this work the inventors describe the preparation and use of 1,3-dioxan-5-one compounds, as fluorescent emitters for organic electroluminescent devices (OLEDs) and for organic light-emitting electrochemical cells (OLECs), with applications that include compounds in the area of phototherapy—including applications to cosmetics.

In the prior art, the fluorescence technology that was utilized displayed a “typical Stokes shift”—wherein an absorption of shorter-wavelength light was followed by a spontaneous emission of light at a longer wavelength. For example, in U.S. Pat. No. 9,726,910, sunlight in the UV or visible region of wavelengths was absorbed and emitted at the longer red and near infrared wavelengths of light. In this prior art of fluorescence-based PBM, a typical Stokes shift—as previously described—meant that a sunglass using this technology would require that the lens be colored, because absorption of visible light was generally required. However, for many consumers who wear prescription lenses, a tinted lens usually corresponds to an expense for a second pair of lenses. A first pair of glasses with a prescription lens usually means a clear lens for most people. For consumers who wish to have UV protections, tint is not an issue because human vision is not responsive to UV light; so if a prescription lens has a UV filter, there is no color imparted to the lens. If, however, a consumer wishes to have a first pair of glasses with a prescription lens that has fluorescence-based PBM according to U.S. Pat. No. 9,726,910, the lens would generally be tinted.

These and other advantages of the present invention will become apparent from the following description and drawings.

### SUMMARY OF THE INVENTION

In the present invention, the physical process of “photon upconversion”—well-known to those skilled in the art—will provide a novel way for a prescription lens to absorb light, for example near infrared and infrared light that is not visible to the human eye, and therefore not impart any tint to the lens, and then fluoresce light in the wavelengths that are shorter and that correspond to the wavelengths of light that are associated with PBM.

More specifically, this invention discloses the use of chemical compounds that are able to undergo photon upconversion type fluorescence wherein light in the infrared region of wavelengths in the range from 850 nm or longer is absorbed by said compound and where photon upconversion type fluorescence emission occurs in the wavelength region from 850 nm to 650 nm. Prescription Lenses made with such compounds will appear colorless, and in the presence of sunlight the lenses will absorb light in the region between 850 nm and 2500 nm and fluoresce in the region of wavelengths—from 850 nm to 650 nm where PBM occurs, thereby providing repair to the eye.

### DETAILED DESCRIPTION OF THE INVENTION

Photon Upconversion is a process where light can be emitted with photon energies higher than those of the light generating the excitation. In this invention it is desired to use special compounds, for example nanometer-sized crystals, that absorb in the infrared or near infrared wavelengths, preferably between 850 nm and 2500 nm and undergo photon upconversion fluorescence and emit in the shorter wavelength regions of the red and near infrared wavelengths associated with photobiomodulation, between 650 nm and 850 nm.

Examples of such compounds include but are not limited to:

1. nanoparticles of yttrium oxyfluoride, ytterbium, erbium/yttrium oxyfluoride (YOF:20% Yb,2% Er/YOF) where strong emission at 669 nm occurs at an excitation wavelength of 980 nm (Guangshun Yi, Chem. Mater. 23, 11, 2729-2734, May 9 2011)
2. Nanoparticles of α-NaYbF₄:Tm³⁺)/CaF₂ where reasonably strong emission at 800 nm occurs at an excitation wavelength of 980 (Guanying Chen, et. al. ACS Nano 2012 6 (9), 8280-8287).

Although some of these dyes—and those described in other references—may have some absorptivity in the visible region of wavelengths, it is possible to utilize those dyes with the highest near IR absorption relative to any absorption in the visible region of wavelengths, and also to dilute the dyes in order to have a high luminous transmission over the visible region of wavelengths, preferably greater than 90% while providing an intensity of fluorescent light greater than 0.1 mW/cm².

Incorporation of the aforementioned nanoparticles into clear, un-tinted, transparent substrates can be achieved by those skilled in the art in a multitude of ways. Including, but not limited to waterborne coatings or solvent based coatings wherein the surface of the nanoparticles are treated or made with hydrophilic or hydrophobic groups respectively. Or the nanoparticles can be dispersed in thermoset resins and cast into lenses or injection molded with lenses.

In the case of skin care products, the nanoparticles—either hydrophobic or hydrophilic—can be co-dispersed in solvents or water with hydrophobic polymers or hydrophilic polymers and then precipitated by a second solvent thereby producing a powder compatible with the skin, wherein the nanoparticle is reasonably sequestered by the powder—and then serve as an ingredient for skin care products. For further sequestration—and further protection to the skin, the nanoparticles can be functionalized so as to co-polymerize with the monomeric form of the aforementioned hydrophobic or hydrophilic polymers—and with the aid of a polymerization initiator.

A summary of mechanisms and materials for fluorescence upconversion—in both organic and inorganic molecules—that may be used as disclosed in this invention can be found in Wikipedia and include but may not be limited to:

1) Inorganic materials that can undergo fluorescence upconversion which contain ions of d-block or f-block elements. Examples are, but not limited to Ln³⁺, Ti²⁺, Ni²⁺, Mo³⁺, Re⁴⁺, Os⁴⁺. The physical mecchanisms for fluorescence upconversion in inorganic materials occurs through energy transfer upconversion, excited state absorption and photon avalanche; and
2) Organic molecules that can undergo fluorescence upconversion by way of triplet-triplet annihilation and usually occur in polycyclicaromatic hydrocarbons.

The above description is illustrative of the preferred embodiment and many modifications may be made by those skilled in the art without departing from the invention whose scope is to be determined from the literal and equivalent scope of the claims below.

What is claimed is:

1. An ophthalmic lens comprising:
a clear, non-tinted, solid plastic substrate; and
a dye suitable for undergoing fluorescence upconversion wherein said dye absorbs light in the near infrared region from 850 nm to 2500 nm and emit light in the shorter wavelength region from 650 nm to 850 nm thereby providing photobiomodulation to the eye.

2. An ophthalmic lens according to claim 1 wherein the substrate is a glass lens.

3. An ophthalmic lens according to claim 1 wherein the substrate is a plastic lens.

4. An ophthalmic lens comprising:
a clear, non-tinted, solid plastic substrate;
a coating or a wafer or a film attached to said solid plastic substrate; and
a dye incorporated into the coating or into the wafer or into the film that is suitable for undergoing fluorescence upconversion wherein said dye absorbs light in the near infrared region from 850 nm to 2500 nm and emit light in the shorter wavelength region from 650 nm to 850 nm thereby providing photobiomodulation to the eye.

5. A cosmetic composition comprising:
a cream or lotion; and
a dye suitable for undergoing fluorescence upconversion wherein said dye absorbs light in the near infrared region from 850 nm to 2500 nm and emit light in the shorter wavelength region from 650 nm to 850 nm thereby providing photobiomodulation to the skin.
